(19) United States
(12) Patent Application Publication
(10) Pub. No.: US 2022/0379042 A1
(43) Pub. Date: Dec. 1, 2022

(54) DISPOSABLE NEEDLE REMOVER AND RECEPTACLE

(71) Applicant: FANJUN ZHOU, NEWTON, MA (US)

(72) Inventor: FANJUN ZHOU, NEWTON, MA (US)

(21) Appl. No.: 17/329,157

(22) Filed: May 25, 2021

(51) Int. Cl. A61M 5/32

(52) U.S. Cl. CPC A61M 5/3278; A61M 2005/3284

(57) ABSTRACT

A device for capturing syringe needles and storing the same includes an upper lid portion mounted tightly onto the lower box portion through an inter-lock system on the opposing sides of the device, and a breaking-twisting mechanism. There is a vertical cylinder inside the upper lid which is centered and sits on a rainbow-like anvil embedded in the lower box. The overall configuration to break a used needle is to insert the syringe needle through the vertical cylinder to hit onto the anvil where the needle is bended, twisted and finally broken off from the syringe through rotating the syringe. The broken needle falls down to the bottom of the lower box under gravity and magnetic force, and is retained there.

### RELATED U.S. APPLICATION DATA

| Int. Cl | A61M5/3278; A61M5/3205; B26F3/00 |
|---|---|
| U.S. Cl | 206/364, 365, 366, 367, 63.5; 83/944 |
| Field of Search | 206/364, 365, 366, 367, 63.5; 83/944; 225/93 |

### REFERENCE CITED

UNITED STATES PATENTS

| | |
|---|---|
| U.S. Pat. No. 3,796,359 March 1974 Peter Dick | 225/103 |
| U.S. Pat. No. 4,375,849 March 1983 Paul Hanifl, et al | 206/365 |
| U.S. Pat. No. 4,553,687 November 1985 Desira Harkins, | 225/93 |
| et al | |
| U.S. Pat. No. 4,969,379 November 1990 Charles Taylor | 83/944 |
| U.S. Pat. No. 9,248,552 B2 February 2016 Josemar Souza | A61M5/3205 |
| dos Santos | |
| U.S. Pat. No. 7.389,873 B2, June 2008 Evelyne Johnson | 206/365 |
| aka Minders | |
| DES. 357,314 April 1995 Joseph Atkins | D24/130 |

### 6 Claims, 3 Drawing Sheets

### BACKGROUND OF THE INVENTION

### 1. Field of the Invention

The present invention relates to the safe disposal of used syringe needles, and also to a miniature device to capture and store the syringe needles.

### 2. Description of the Related Art

Disposable syringes are being used widely in many occasions and are getting more universal. Syringe needles with drug and medical residues could possibly cause the spread of infections like Hepatitis B (HBV) and Hepatitis C (HCV) and affect human health conditions seriously. In recognition of the risk above, the safety devices or safely disposal of the disposable syringe needles is mandatory in the US. Syringes with auto-disable features or capping mechanism have been developed.

There have been needle removers that destruct needles electrically or mechanically. With such a device, users can safely dispose of a large number of used syringe needles, and it is removed for independent disposal after it is full. Additionally, portable devices to remove and store the needle were also developed. F. e. U.S. Pat. No. 7,389,873 B2 described a pocket-sized remover and receptacle whereby needles and the syringe were broken with a guillotine-like mechanism. However, the configurations of the syringe bottom are various, and it is actually hard to well accommodate the syringe into the device opening to cut the needle short enough to prevent the stubble from being further harmful. Also, mounting the metal guillotine onto the plastic box presents another practical difficulty for a cigarette lighter size device. Consequently, it's desirable to have a small, portable device that provides convenience to cut the needle short enough and store it while at the same time it's easy for users to carry around and even for syringe manufacturers to directly put the syringe in their kits inexpensively.

### SUMMARY OF THE INVENTION

A device for capturing used syringe needles has a lower box portion and an upper lid portion tightly connected with each other through an interlock system located on the opposing sides of device. Furthermore, there is a vertical cylinder inside the upper lid which is centered on a rainbow-like anvil mounted inside the lower box. The disposable syringe needle after its use is pushed through the cylinder, bended at its root on the surface of the anvil, and then twisted off through rotating the syringe.

The mechanism to sever the needle off the syringe is a combination of breaking (bending the needle almost to the right angle at its root position) and twisting (along the circumferential direction). The bending of the needle generates great fractures, especially at its root position of the needle, which tremendously weakens the needle from the syringe. Further separation of needle from the syringe can be easily achieved through swiping the needle along circular direction while rotating the syringe.

Preferably the anvil has a rainbow-like shape, forming an arc along its longitudinal direction. The arc shape can direct the needle to extend in longitudinal direction when it is bended on the surface of the anvil. Also, with the cylinder above it, there are openings between the anvil and cylinder in the longitudinal direction but close contacts in the lateral direction (“joining points”). Preferably the openings are big enough for the needle to go through when bended and extended, and at the same time, small enough to prevent the broken needles from aligning with cylinder and dropping out of the device. Preferably the anvil has some projected ridges along its longitudinal direction, which combines with the jointing points between the anvil and the cylinder, provide effective multiple means to swipe off the weakened needles from the syringe when rotating the syringe. Also, preferably there is a magnet at the bottom of the box, keeping the broken needles from escaping the device. The magnet sits in a horizontal cylinder at the anvil base and is fixed by two opposing poles.

### BRIEF DESCRIPTION OF THE SEVERAL VIEWS OF THE DRAWINGS

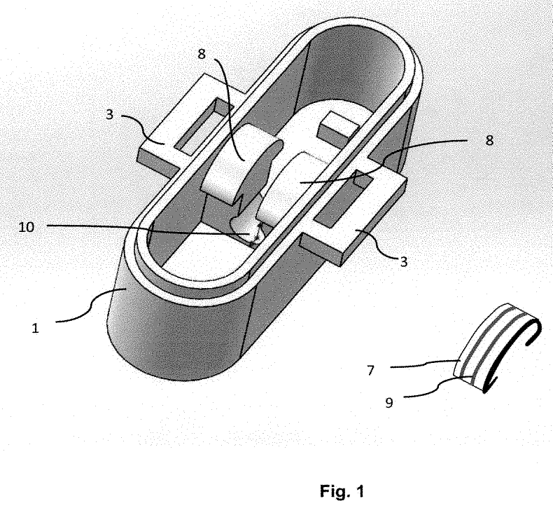

FIG. 1 is a perspective three-dimensional view of the lower box portion of the needle remover's preferred embodiment. The inset at the right bottom corner is the perspective view of the anvil, which is mounted on a slitted base by the inward-rolled hooks on opposing sides.

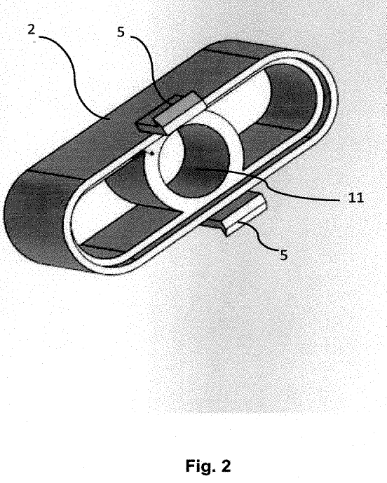

FIG. 2 is a perspective three-dimensional view of the upper lid portion of the needle remover. The needle is pushed through the cylinder, hit onto the anil, bended and twisted off the syringe.

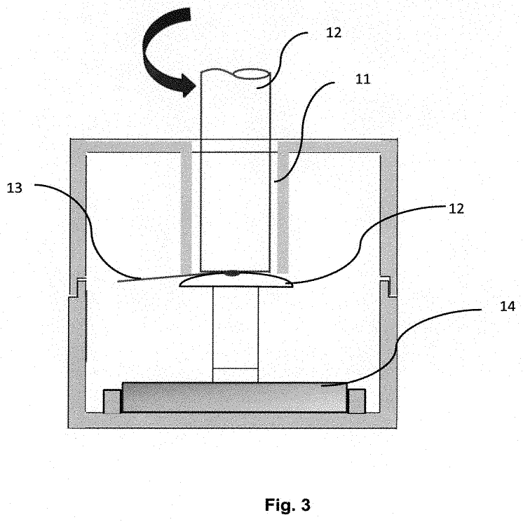

FIG. 3 is a view showing the needle being bended on the anvil.

### DESCRIPTION OF THE PREFERRED EMBODIMENT

FIGS. 1 & 2 show a pocket-sized needle remover and receptacle of the present invention's preferred embodiment. The device includes a lower box portion 1 and an upper lid portion 2 that are interlocked together through a conventional plug-in system 3 and 5 lying on the opposing sides of the device. Preferably this interlock system is protruded high enough above the surface so that the user's hand can hold the receptacle behind it to prevent from being hurt by a needle. The inset in FIG. 1 shows a view of the anvil 7, which is mounted on its slitted base 8 by two inward-rolled hooks on the two opposing sides. The anvil 7 is made of iron, steel, porcelain or any hard materials, on which the needle can be collided, bended and twisted instead of being easily pierced through. Preferably the anvil has a rainbow-like shape to direct the needle to go along its longitudinal direction when bended. Preferably the openings between the anvil and the cylinder are big enough for the needle to go through when being bended and extended, and at the same time, small enough to prevent the broken needles from aligning with cylinder and dropping out of the device. Also, preferably the anvil has some projected ridges 9 along the longitudinal direction. These grooves, together with the jointing points between the anvil and the cylinder, can provide effective blocks to swipe off the needles from the syringe when the syringe is rotated. Preferably there is a horizontal cylinder 10 at the bottom of the anvil base, where a magnetic is mounted to attract the broken needles. FIG. 2 depicts a perspective view of the vertical cylinder of the device's upper lid portion 2, which after mounting, sits right on the center of the anvil 7. The needle after its use is directed through the cylinder to hit onto the anvil, and then bended and twisted off the syringe. Preferably there is a conventional protective cover (not shown) to cover the cylinder outside to further prevent the broken needles from escaping out of the box.

FIG. 3 describes the operation and function of the device. The device is handy, portable and can be easily put into a pocket, purse, supplies kit, etc. A disposable syringe 12 after its use is held in one hand, and the needle portion 13 inserted through the cylinder 11. After the needle reaches the anvil 7, firmly push down the syringe 11 against the anvil 7 until the needle 11 is bended to almost the right angle at its root position. Then, with a few fingers, the user rotates the syringe 11 on the anvil 7 to twist the needle 13 off the syringe 11 when it is brought contact with either the anvil ridges or at the jointing points between the anvil 7 and the cylinder 11. As the needle 13 is broken, it falls under gravity and magnetic force into the bottom of the box and is retained there.

While the present invention has been embodied here, additional variations of the present invention may be devised based on the inventive conceptive.

What is claimed is:

1. A device for securely capturing and storing needles comprising:
an upper lid portion with a vertical cylinder inside;
a lower box portion interlocked with the upper lid portion;
an anvil mounted inside the lower box portion.

2. The device of claim 1 wherein the upper lid and lower box portions are interconnected and sealed by a plug-in system.

3. The device of claim 1 wherein the anvil has a rainbow-like shape to direct the needle to go along the longitudinal direction when bended.

4. The device of claim 3 wherein the openings between the anvil and the cylinder are big enough for the needle to go through when bended and extended, and at the same time, small enough to prevent the broken needles from aligning with cylinder and dropping out of the device.

5. The device of claim 3 wherein the arc anvil surface has some projected ridges along the longitudinal direction to provide effective blocks to swipe off the needles from the syringe when the syringe is rotated.Δ

6. A method of removing and storing needles comprising the following steps:
Providing a vertical cylinder, an anvil right below the cylinder and with blocking ridges and receptacle box;
Inserting a syringe needle through the vertical cylinder;
Forcibly pushing the syringe against the anvil thereby bending the needles, and then rotating the syringe on the anvil thereby the blocking ridges swipe the needles off the syringe.
